(19) United States
(12) Patent Application Publication
(10) Pub. No.: US 2009/0305246 A1
(43) Pub. Date: Dec. 10, 2009

(54) SCHIZOPHRENIA ASSOCIATED GENES AND MARKERS

(76) Inventors: Bernard Lerer, Alon Shvut (IL); Daniela Zalcenstein, Rehovot (IL); Kyra Kanyas, Moshav Beit Zayit (IL)

Correspondence Address: WINSTON & STRAWN LLP;PATENT DEPARTMENT, 1700 K STREET, N.W., WASHINGTON, DC, 20006, US

(21) Appl. No.: 12/090,187

(22) PCT Filed: Oct. 17, 2006
(86) PCT No.: PCT/IL06/01195

§ 371 Date: Jul. 16, 2008

Related U.S. Application Data
(60) Provisional application No. 60/726,605, filed on Oct. 17, 2005.

(51) Int. Cl. C12Q 1/68; C07H 21/04

(52) U.S. Cl. 435/6; 536/24.31

(57) ABSTRACT

The invention discloses schizophrenia-associated polymorphism located on chromosome 6q23 within the human Abelson Helper Integration Site 1 gene (AHI1), or a genomic region linked to the AHI1 gene that includes the C6orf217 gene. The invention further discloses systems and methods for diagnosing schizophrenia or predisposition to schizophrenia.

### FIELD OF THE INVENTION

The present invention relates to schizophrenia-associated polymorphism within the human Abelson Helper Integration Site 1 gene (AHI1), or a genomic region linked to the AHI1 gene comprising the C6orf217 gene, and to methods and means for diagnosing schizophrenia or predisposition to schizophrenia.

### BACKGROUND OF THE INVENTION

Schizophrenia (OMIM Database, MIM 181500) is a severe neuropsychiatric disorder that has its overt onset in late adolescence or early adulthood. The disease is clinically characterized by a variety of symptoms, including “positive” symptoms such as delusions, hallucinations and thought disorder that tend to occur episodically and “negative” symptoms that are relatively persistent, including lack of drive and motivation, poor social and occupational adjustment and cognitive dysfunction (primarily impairment of executive functions). Schizophrenia, affecting approximately 1% of the population, is the leading cause of chronic psychiatric hospitalization worldwide and represents a major public health and economic burden. While the etiology of schizophrenia is not known, a significant body of evidence supports a neurodevelopmental model, which suggests a pivotal role for abnormalities of brain development in utero and post-natally (Rapoport J L, et al. 2005. Mol Psychiatry 10:434-449).

It has long been hypothesized that genetic factors play a significant role in schizophrenia with strong support from family, twin and adoption studies (for example Lichtermann D, et al. 2000. Eur Arch Psychiatry Clin Neurosci 250:304-310). Inheritance studies revealed that it is a multi-factorial disease characterized by a multiple genetic susceptibility elements, each is likely to contribute a modest increase in risk. Although linkage studies aimed at mapping schizophrenia susceptibility loci have not been fully consistent, there are indications for certain chromosomal regions being associated with the disorder. Further efforts have focused on identifying susceptibility genes, mostly in regions previously implicated by linkage studies, identifying the genes dysbindin (DTNBP1), neuregulin1 (NRG1), D-amino acid oxidase activator (DAOA, previously known as G72), D-amino acid oxidase (DAAO), catechol-O-methyltransferase (COMT, WO 03/070082), regulator of G-protein signaling 4 (RGS4) disrupted-in-schizophrenia-1 (DISC1) and proline dehydrogenase PRODH to be associated with schizophrenia. While these genes have potential pathophysiological relevance to schizophrenia and in some cases a putative role in brain development, pathogenic mutations or genetic variants that influence function by other mechanisms have not yet been identified. The possibility remains that genes in linkage disequilibrium with these loci are in fact implicated and that genes in other regions may be involved in the pathophysiology of the disorder.

Recently, considerable interest has focused on the long arm of chromosome 6, where several studies have mapped putative schizophrenia susceptibility loci. Following the report of Cao et al. (1997. Genomics 43:1-8), who found excess allele sharing for markers in the 6q13-26 region, other linkage studies, based on samples of varying size and ethnicity supported this finding. In the met-analysis of Badner and Gershon (2002. Mol Psychiatry 7:405-411) chromosome 6q met the significance criterion of the meta-analysis but not the criterion for replication. A further development of considerable interest is that several groups have reported evidence for linkage of bipolar disorder on chromosome 6q and linkage of psychosis in bipolar pedigrees (Park N. et al, 2004. Mol Psychiatry 9:1091-1099). Given the extensive genomic distance spanned by these reports, it is feasible that the chromosome 6q region harbors more that one gene implicated in the pathogenesis of schizophrenia, bipolar disorder and possibly other neuropsychiatric phenotypes.

International PCT Application WO 2006/023719 discloses the association of a gene known as TRAR4 located on chromosome 6q13-q26, coding for a receptor, with schizophrenia and schizoaffective disorders.

A paper by the inventor of the present invention published after the priority date of the present application describes a linkage between the Abelson Helper Integration Site 1 (AHI1) gene and an adjacent, primate-specific gene C6orf217 with schizophrenia (Amann-Zalcenstein D. et al, 2006. Eur J Hum Genet 14(10):1111-1119). These two genes appear in opposite orientations and their regulatory upstream regions overlap, which might affect their expression.

During evolution of any organism, mutations occur and generate variant forms of progenitor sequences. When a variant form confers an evolutionary advantage to the species, it is inherited to the next generations. When the evolutionary advantage is significant, the variant may incorporate into the DNA of many or most members of the species, such that the variant becomes the progenitor form. In many instances, both progenitor and variant form(s) survive and co-exist in a species population. This coexistence of multiple forms of a sequence gives rise to polymorphisms.

Several different types of polymorphism have been reported. A restriction fragment length polymorphism (RFLP) means a variation in DNA sequence that alters the length of a restriction fragment as described, for example, in Botstein et al. (1980. Am J Hum Genet 32:314-331). The restriction fragment length polymorphism may create or delete a restriction site, thus changing the length of the restriction fragment. When a heritable trait can be linked to a particular RFLP, the presence of the RFLP in an individual can be used to predict the likelihood that the individual will also exhibit the trait. Other polymorphisms take the form of short tandem repeats (STRs) that include tandem di-, tri- and tetranucleotide repeated motifs. These tandem repeats are also referred to as variable number tandem repeat (VNTR) polymorphisms. VNTRs have been used in identity and paternity analysis, and in a large number of genetic mapping studies.

Other forms of polymorphism include single nucleotide variations between individuals of the same species. Such polymorphism is far more frequent than RFLPs, STRs and VNTRs, and a single nucleotide polymorphism (SNP) may also result in a RFLP because a single nucleotide change can also result in the creation or destruction of a restriction enzyme site. Some single nucleotide polymorphisms occur in protein-coding sequences, in which case, one of the polymorphic forms may give rise to the expression of a defective or other variant protein and, potentially, a genetic disease. Examples of genes, in which polymorphism within coding sequences give rise to genetic diseases, include beta-globin (sickle cell anemia) and cystic fibrosis (CFTR). Single nucleotide polymorphisms that occur in noncoding regions may also result in defective protein expression, for example as a result of alternative splicing or quantitative and other effects on gene expression. Other single nucleotide polymorphisms have no known phenotypic effects but may be genetically linked to a phenotypic effect by as yet undefined mechanisms.

The greater frequency and uniformity of single nucleotide polymorphism means that there is a greater probability that such a polymorphism will be found in close proximity to a genetic locus of interest than would be the case for other polymorphisms. Also, the different forms of characterized single nucleotide polymorphisms are often easier to distinguish than other types of polymorphism (e.g., by use of assays employing allele-specific hybridization probes or primers). In a disease such as schizophrenia in which multiple gene products play a role in the analysis of the disease, SNPs show particular promise as a research tool, and they may also be valuable diagnostic tools.

Based on linkage and association studies described above, a large number of schizophrenia associated genes and SNPs were identified; the following references are merely representative.

U.S. Patent Application Publication Nos. 200301070667 and 20040115699 disclose nucleic acid segments of the human G protein coupled receptor Seq-40 gene including polymorphic sites, and provide allele specific primers and probes hybridizing to regions flanking these sites and methods for determining the genetic risk of developing schizophrenia or diagnosing schizophrenia. Similarly, U.S. patent Application 20030224365 provides nucleic acid segments of the human G protein coupled receptor Con-202 gene including polymorphic sites and methods of use thereof for determining the genetic risk of developing schizophrenia or diagnosing schizophrenia.

U.S. Patent Application Publication No. 20030219750 relates to the association established between schizophrenia and bipolar disorder and biallelic markers identified within the sbg1, g34665, sbg2, g35017 and g35018 genes and nucleotide sequences. That application discloses means to identify compounds useful in the treatment of schizophrenia, bipolar disorder and related diseases, means to determine the predisposition of individuals to said disease as well as means for the disease diagnosis and prognosis.

U.S. Patent Application No. 20040014095 provides methods for the diagnosis of schizophrenia and susceptibility to schizophrenia by detection of polymorphisms, mutations, variations, alterations in expression, etc., in calcineurin genes or calcineurin interacting genes, or polymorphisms linked to such genes. This application discloses methods for detection of polymorphisms and variants, methods of treating schizophrenia by administering compounds that target these genes, screening methods for identifying such compounds and compounds obtained by performing the screens.

International Application WO 2005/004702 discloses methods for the diagnosis of schizophrenia and susceptibility to schizophrenia by detection of polymorphisms, mutations, variations, alterations in expression, etc., in genes encoding an early growth response (EGR) molecule or an EGR interacting molecule, or polymorphisms linked to such genes. The invention also discloses methods of treating schizophrenia by administering compounds that target these genes, and screening methods for identifying such compounds and compounds obtained by performing the screens.

The inventors of the present invention and co-workers previously reported linkage of schizophrenia to chromosome 6q23 (Lerer B. et al, 2003. Mol Psychiatry 8:488-498). However, this large chromosomal region can contain a large number of genes, most of them probably not related to schizophrenia. Thus, there is a recognized need for, and it would be highly advantageous to have specific genetic markers for the diagnosis and prognosis of schizophrenia as well as for predicting a predisposition to develop schizophrenia.

### SUMMARY OF THE INVENTION

The present invention relates to the discovery of the association of schizophrenia with the Abelson Helper Integration Site 1 (AHI1) gene and a linked neighboring non-annotated gene of unknown function. The invention also relates to the identification of a set of schizophrenia-related polymorphic markers. The present invention further relates to use of the polymorphic markers as targets for diagnosis of schizophrenia and susceptibility to schizophrenia.

Based on the linkage of schizophrenia to chromosome 6q23, previously described by the inventors, the present invention now discloses a novel set of single nucleotide polymorphisms (SNPs) within the AHI1 gene with additional SNPs extending to the adjacent phosphodiesterase 7B (PDE7B) gene and the intergenic region which includes a putative intervening gene of unknown function (C6orf217). This discovery associates the AHI1 gene, and potentially the C6orf217 gene with the pathogenesis of schizophrenia and related conditions.

According to one aspect, the present invention provides a method for diagnosing schizophrenia or predisposition to schizophrenia in a subject comprising (a) obtaining a sample comprising genetic material from the subject; (b) determining, in the genetic material, the nucleic acid sequence within a gene encoding AHI1 protein or a genomic region linked to the AHI1 gene comprising a gene designated C6orf217; and (c) analyzing said nucleic acid sequence for polymorphism indicative of schizophrenia or a predisposition to schizophrenia.

According to one embodiment, analyzing the nucleic acid sequence for polymorphism comprises determining the identity of at least one polymorphic site within the AHI1 gene having a reference sequence number on chromosome 6q23 selected from the group consisting of rs6931735, rs6912933, rs9321501, rs2746429, rs2614258 and rs11154801. In one embodiment, the presence of A (Adenine) at rs6931735, rs6912933 or rs9321501; T (Thymine) at rs2746429; G (Guanine) at rs2614258 or C (Cytosine) at rs11154801, indicates that the subject has schizophrenia or predisposition to schizophrenia.

According to another embodiment, analyzing the nucleic acid sequence for polymorphism comprises determining the identity of at least one polymorphic site within the gene designated C6orf217 having a reference sequence number on chromosome 6q23 selected from the group consisting of rs7750586, rs9647635, rs7739635 and rs9494332. In one embodiment, the presence of A at rs7750586 or rs9647635; C at rs7739635; or G at rs9494332 indicates that the subject has schizophrenia or predisposition to schizophrenia.

According to yet another embodiment, analyzing the nucleic acid sequence for polymorphism comprises determining the identity of a polymorphic site within a genomic region between the gene designated C6orf217 and the PDE7B gene having a reference sequence number rs1475069 on chromosome 6q23. In one embodiment, the presence of A at rs1475069 indicates that the subject has schizophrenia or predisposition to schizophrenia.

According to certain embodiments, analyzing the nucleic acid sequence for polymorphism comprises determining the identity of a polymorphic site within a gene encoding FAM54A protein having a reference sequence number rs797553 on chromosome 6q23. In one embodiment, the presence of G at rs797553 indicates that the subject has schizophrenia or predisposition to schizophrenia.

According to other embodiments, analyzing the nucleic acid sequence for polymorphism comprises determining the identity of at least one polymorphic site at about 140-142 Mb on chromosome 6q23 having a reference sequence number selected from the group consisting of rs642162, rs1414839 and rs1239365. In one embodiment, the presence of C at rs642162; rs1414839; or rs1239365 indicates that the subject has schizophrenia or predisposition to schizophrenia.

According to another aspect, the present invention provides a method for diagnosing schizophrenia or predisposition to schizophrenia in a subject comprising (a) obtaining a sample comprising genetic material from the subject; (b) determining, in the genomic material, the nucleic acid sequence within a gene encoding AHI1 protein or a genomic region linked to the AHI1 gene comprising a gene designated C6orf217; and (c) analyzing said nucleic acid sequence for the presence of at least one haplotype; wherein the presence of at least one schizophrenia-associated haplotype is indicative of schizophrenia or predisposition to schizophrenia.

According to one embodiment, the schizophrenia-associate haplotype comprises at least two polymorphic sites having reference sequence number selected from the group consisting of rs878175, rs6931735, rs6912933, rs9321501, rs2746429 and rs2614258.

According to one currently preferred embodiment, the schizophrenia-associated haplotype comprises polymorphic sites having reference sequence number rs878175, rs6931735 and rs6912933 on chromosome 6q23. The haplotype indicates schizophrenia or predisposition to schizophrenia wherein the nucleotide identity at the reference sequence numbers rs878175, rs6931735 and rs6912933 is TGG respectively.

According to another currently preferred embodiment, the schizophrenia-associated haplotype comprises polymorphic sites having reference sequence number rs6931735, rs6912933, and rs9321501 on chromosome 6q23. The haplotype indicates schizophrenia or predisposition to schizophrenia wherein the nucleotide identity at the reference sequence numbers rs6931735, rs6912933, and rs9321501, respectively, is selected from the group consisting of AAA and GGC.

According to yet another currently preferred embodiment, the schizophrenia-associated haplotype comprises polymorphic sites having reference sequence number rs6912933, rs9321501 and rs2746429 on chromosome 6q23. The haplotype indicates schizophrenia or predisposition to schizophrenia wherein the nucleotide identity at the reference sequence numbers rs6912933, rs9321501 and rs2746429, respectively, is selected from the group consisting of AAT and GCC.

According to a further currently preferred embodiment, the schizophrenia-associated haplotype comprises polymorphic sites having a reference sequence number rs9321501, rs2746429 and rs2614258 on chromosome 6q23. The haplotype indicates schizophrenia or predisposition to schizophrenia wherein the nucleotide identity at the reference sequence numbers rs9321501, rs2746429 and rs2614258, respectively, is selected from the group consisting of ATG and CCA.

According to further aspect, the present invention provides a method for diagnosing schizophrenia or predisposition to schizophrenia in a subject comprising (a) obtaining a sample comprising genetic material from the subject; (b) determining, in the genetic material, the nucleic acid sequence within a genomic region between the IL22RA2 gene and the TNFAIP3 gene at about 138.1 Mb on chromosome 6q23; and c) analyzing said nucleic acid sequence for the presence of at least one haplotype; wherein the presence of at least one schizophrenia-associated haplotype is indicative of schizophrenia or predisposition to schizophrenia.

According to one embodiment, the schizophrenia-associated haplotype comprises at least two polymorphic sites having reference sequence number selected from the group consisting of rs667520, rs999638 and rs683122 on chromosome 6q23.

According to one currently preferred embodiment, the schizophrenia-associated haplotype comprises polymorphic sites having reference sequence number rs667520, rs999638 and rs683122 on chromosome 6q23. The haplotype indicates schizophrenia or predisposition to schizophrenia wherein the nucleotide identity at the reference sequence numbers rs667520, rs999638 and rs683122 is ATA respectively.

According to still further aspect, the present invention provides a method for diagnosing schizophrenia or predisposition to schizophrenia in a subject comprising (a) obtaining a sample comprising genetic material from the subject; (b) determining, in the genetic material, the nucleic acid sequence within the C6orf55 gene and the REPS1 gene at about 139 Mb on chromosome 6q23; and c) analyzing said nucleic acid sequence for the presence of at least one haplotype; wherein the presence of at least one schizophrenia-associated haplotype is indicative of schizophrenia or predisposition to schizophrenia.

According to one embodiment, the schizophrenia-associated haplotype comprises at least two polymorphic sites having reference sequence number selected from the group consisting of rs2876391, rs1188852 and rs1188863 on chromosome 6q23.

According to one currently preferred embodiment, the schizophrenia-associated haplotype comprises polymorphic sites having reference sequence number rs2876391, rs1188852 and rs1188863 on chromosome 6q23. The haplotype indicates schizophrenia or predisposition to schizophrenia wherein the nucleotide identity at the reference sequence numbers rs2876391, rs1188852 and rs1188863 is TAA respectively.

Any method for determining nucleic acid sequence and for analyzing the identified nucleotides for polymorphism, known to a person skilled in the art, can be used according to the teachings of the present invention.

According to certain embodiments, detecting the presence of at least one nucleotide polymorphism is performed by a technique selected from the group consisting of: terminator sequencing, restriction digestion, allele-specific polymerase reaction, single-stranded conformational polymorphism analysis, genetic bit analysis, temperature gradient gel electrophoresis ligase chain reaction and ligase/polymerase genetic bit analysis.

According to other embodiments, the nucleotide polymorphism is detected by employing nucleotides with a detectable characteristic selected from the group consisting of inherent mass, electric charge, electric spin, mass tag, radioactive isotope type bioluminescent molecule, chemiluminescent molecule, nucleic acid molecule, hapten molecule, protein molecule, light scattering/phase shifting molecule and fluorescent molecule.

According to an additional aspect, the present invention provides an isolated polynucleotide designed to specifically detect a naturally occurring polymorphic variant of a polymorphism indicative of schizophrenia or predisposition to schizophrenia within a region on chromosome 6q23 from about 136 Mb to about 142 Mb. According to one embodiment, the isolated polynucleotide comprises from about 10 to about 100 contiguous nucleotides, preferably from about 15 to about 30 contiguous nucleotides, wherein the polynucleotide is designed to specifically hybridize to a nucleic acid molecule comprising at least one polymorphic site of the polymorphism indicative of schizophrenia or predisposition to schizophrenia according to the present invention.

According to one currently preferred embodiment, the isolated polynucleotide is designed to specifically amplify a segment of chromosome 6q23 comprising at least one polymorphic site of the polymorphism indicative of schizophrenia or predisposition to schizophrenia according to the present invention.

According to certain embodiments, the amplified segments of chromosome 6q23 starts at least 15 and typically not more than 100 nucleotides from the polymorphic site indicative for schizophrenia or predisposition to schizophrenia. According to one embodiment, the amplified segment comprises from about 80 to about 200 contiguous nucleotides.

Other objects, features and advantages of the present invention will become clear from the following description and drawings.

### BRIEF DESCRIPTION OF THE FIGURES

FIG. 1 shows SNP distribution on a schizophrenia-susceptibility-locus of 13.9 Mb in a genomic region of chromosome 6q23, found in an Arab-Israeli cohort. FIG. 1A: Multipoint, non-parametric linkage analysis of microsatellite markers on chromosome 6q under broad diagnostic model showing a maximum NPL of 4.98 (p=0.00000058) at 136.97 cM. The NPL-1 (3.9 Mb) and NPL-2 (˜20 Mb) confidence intervals are indicated by broken lines. The linkage peak is represented by a triangle. FIG. 1B: The NPL-1 and NPL-2 confidence intervals are indicated by horizontal arrows. The position of the linkage peak is depicted by a triangle at 136.3 Mb. Distribution of known genes spanning a ˜14 Mb genomic region underneath the linkage peak is shown. Gene polarity is indicated by the orientation of the horizontal triangles. Dark and light triangles represent, respectively, genes covered by the genotyped SNPs or devoid of genotyped SNPs. The scissors represent known recombination hotspots. Genes covered by genotyped SNPs are: a: TRAR4, b: MYB, c: AHI1 d: C6orf217, e: PDE7B, f: FAM54A, g: BCL2A, h: MAP7, i: MAP3K5, j: PEX7, k: IL22RA2, l: TNFAIP3, m: C6orf63, n: REPS1, o: HECA, p: NMBR, q: C6orf55, r: GPR126, s: EPM2A, t: GRM1. FIG. 1C: Distribution of SNPs within the 14 Mb genomic region under the linkage peak. Dark vertical lines indicate dense clusters of SNPs. The SNP density is highest in a ˜1 Mb region from 135.5 to 136.5 Mb with an average inter-SNP distance of 17.0 kb, followed by the ˜7 Mb genomic region from 136.5 to 143.5 Mb with an average inter-SNP distance of 66.6 kb. The remaining SNPs are distributed across 3 candidate gene, namely TRAR4 (a), EPM2A (s) and GRM1 (t). Position of 23 haplotype blocks relative to the LD plot are shown underneath, as well as the LD plot generated using HAPLOVIEW software with pairwise SNP comparison for SNPs less than 1 Mb apart. Dark area indicates regions of high LD and white areas represent regions of low LD.

FIG. 2 shows single SNP association results within the NPL-2 confidence interval of a linkage peak on chromosome 6q23 of a schizophrenia-susceptibility-locus in an Arab-Israeli cohort (the SNP number is an arbitrary number). The broken line represents the Bonferroni cut-off for multiple testing (p=0.00028). The significant SNP cluster from SNP No. 17 to No. 41 spans from the MYB gene to C6orf217.

FIG. 3 shows haplotype analysis across the 13.9 Mb genomic region on chromosome 6q using 3-SNP sliding windows (the haplotype number is an arbitrary number). The broken line represents the Bonferroni cut-off for multiple testing (p=0.000082). The most significant haplotype cluster around haplotype No. 53 is located within the AHI1 gene.

### DETAILED DESCRIPTION OF THE INVENTION

### Definitions

As used herein, the term “gene” has its meaning as understood in the art. In general, a gene is taken to include gene regulatory sequences (e.g. promoters, enhancers, etc.) and/or intron sequences, in addition to coding sequences (open reading frames). It will further be appreciated that definitions of “gene” include references to nucleic acids that do not encode proteins but rather encode functional RNA molecules such as microRNAs (miRNAs), tRNAs, etc. For the purpose of clarity it is noted that, as used in the present application, the term “gene” generally refers to a portion of a nucleic acid that encodes a protein; the term may optionally encompass regulatory sequences. This definition is not intended to exclude application of the term “gene to non-protein coding expression units but rather to clarify that, in most cases, the term as used in this document refers to a protein coding nucleic acid.

The term “allele” as used herein refers to one of the different forms of a gene or DNA sequence that can exist at a single locus within the genome.

The terms “complementary” or “complement thereof” are used herein to refer to the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

The term “genotype” as used herein refers to the identity of the alleles present in an individual or a sample. In the context of the present invention a genotype preferably refers to the description of the polymorphic alleles present in an individual or a sample. The term “genotyping” a sample or an individual for a polymorphic marker refers to determining the specific allele or the specific nucleotide sequence carried by an individual at a polymorphic marker.

The term “haplotype” refers to the actual combination of alleles on one chromosome. In the context of the present invention a haplotype preferably refers to a combination of polymorphisms found in a given individual and which may be associated with a phenotype.

The term “polymorphism” as used herein refers to the occurrence of two or more alternative genomic sequences or alleles in a population. “Polymorphic” refers to the condition in which two or more variants of a specific genomic sequence can be found in a population. A “polymorphic site” is the locus at which the variation occurs. Polymorphism refers to the occurrence of two or more genetically determined alternative sequences or alleles in a population. Preferred polymorphisms have at least two alleles, each occurring at frequency of greater than 1%, and more preferably greater than 10% or 20% of a selected population. A polymorphic locus may be as small as one base pair. Polymorphic markers include restriction fragment length polymorphisms, variable number of tandem repeats (VNTRs), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wild type form. Diploid organisms may be homozygous or heterozygous for allelic forms. A biallelic polymorphism has two forms. A triallelic polymorphism has three forms.

A “single nucleotide polymorphism” (SNP) is a single base pair change. A single nucleotide polymorphism occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations).

A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphism can also arise from a deletion of a nucleotide or an insertion of a nucleotide relative to a reference allele. It should be noted that a single nucleotide change could result in the destruction or creation of a restriction site. Therefore it is possible that a single nucleotide polymorphism might also present itself as a restriction fragment length polymorphism.

Single nucleotide polymorphisms (SNPs) can be used in the same manner as RFLPs and VNTRs but offer several advantages. Single nucleotide polymorphisms occur with greater frequency and are spaced more uniformly throughout the genome than other forms of polymorphism. SNPs occur at a frequency of roughly 1/1000 base pairs, and are distinguished from rare variations or mutations by a requirement for the least abundant allele to have a frequency of 1% or more. Examples of SNP include non-synonymous coding region changes which substitute one amino acid for another in the protein product encoded by the gene; synonymous changes which do not alter amino acid coding sequence due to degeneracy of the genetic code; changes in promoter, enhancer or other genetic control element sequence which may or may not alter transcription of the gene; changes in untranslated regions of the mRNA, particularly at the 5′ end which may alter the efficiency of ribosomal binding, initiation or translation, or at the 3′end which may alter mRNA stability; and changes within intronic regions which may alter the splicing of the transcript or the function of other genetic regulatory elements.

The terms “polymorphism within AHI1 gene or a genomic region linked to AHI1 gene” or “AHI1 polymorphic site” or “polymorphic site within a genomic region linked to the AHI1 gene” are used herein to mean a polymorphism or polymorphic site within about 1 Mb genomic region around the linkage peak of schizophrenia susceptibility locus on chromosome 6q23 (Lerer B. et al. 2003. supra; Levi A. et al. 2005. Eur J Hum Genet 13:763-71). Five known genes reside in this ˜1 Mb genomic region, namely MYB, AHI1, PDE7B, FAM54A and BCL2A, as well as a gene of unknown function, C6orf217, for which there is an EST evidence. This term would encompass polymorphisms at polymorphic sites within the gene coding sequences, intronic regions and flanking regions. A polymorphism according to the present invention may or may not change an amino acid in the protein product of the genes, specifically the AHI1 gene, in order to have utility. The term polymorphism within AHI1 gene or a genomic region linked to AHI1 gene encompasses single nucleotide polymorphisms, biallelic and otherwise and include the polymorphisms described in Table 1 hereinbelow. The term “at least one polymorphic site” means at least one polymorphic site within the above described about 1 Mb genomic region having a reference sequence number as disclosed herein.

As used interchangeably herein, the term “oligonucleotides”, and “polynucleotides” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. The term “nucleotide” is also used herein to encompass “modified nucleotides” which comprise at least one modifications, including, for example, analogous linking groups, purine, pyrimidines, and sugars. However, the polynucleotides of the invention are preferably comprised of greater than 50% conventional deoxyribose nucleotides, and most preferably greater than 90% conventional deoxyribose nucleotides The polynucleotide sequences of the invention may be prepared by any known method, including synthetic, recombinant, ex vivo generation, or a combination thereof, as well as utilizing any purification methods known in the art.

The term “linkage disequilibrium”, or LD, is the non-random association of alleles at two or more loci. It is not the same as linkage, which describes the association of two or more loci on a chromosome with random recombination between them. LD describes a situation in which some combinations of alleles or genetic markers occur more or less frequently in a population than would be expected from a random formation of haplotypes from alleles based on their frequencies. Linkage disequilibrium is typically caused by fitness interactions between genes or by such non-adaptive processes as population structure, inbreeding, and stochastic effects. In population genetics, linkage disequilibrium is said to characterize the haplotype distribution at two or more loci.

As used herein, a sample comprising genetic material obtained from a subject may include, but is not limited to, any or all of the following: a cell or cells, a portion of tissue, blood, serum, ascites, urine, saliva, amniotic fluid, cerebrospinal fluid, and other body fluids, secretions, or excretions. The sample may be a tissue sample obtained, for example, from skin, muscle, buccal or conjunctival mucosa, placenta, gastrointestinal tract or other organs. A sample of DNA from fetal or embryonic cells or tissue can be obtained by appropriate methods, such as by amniocentesis or chorionic villus sampling.

As used herein, the term “isolated” means 1) separated from at least some of the components with which it is usually associated in nature; 2) prepared or purified by a process that involves the hand of man; and/or 3) not occurring in nature. Particularly, the term is used herein to describe a polynucleotide of the invention which has been to some extent separated from other compounds including, but not limited to other nucleic acids, carbohydrates, lipids and proteins (such as the enzymes used in the synthesis of the polynucleotide), or the separation of covalently closed polynucleotides from linear polynucleotides. A polynucleotide is substantially isolated when at least about 50%, preferably 60 to 75% of a sample exhibits a single polynucleotide sequence and conformation (linear versus covalently closed). The degree of polynucleotide isolation or homogeneity may be indicated by a number of means well known in the art, such as agarose or polyacrylamide gel electrophoresis of a sample, followed by visualizing a single polynucleotide band upon staining the gel. For certain purposes higher resolution can be provided by using HPLC or other means well known in the art.

The term primer refers to a single-stranded oligonucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically ranges from 15 to 30 nucleotides. Short primer molecules generally require cooler temperatures to form sufficiently stable hybrid complexes with the template. A primer need not reflect the exact sequence of the template but must be sufficiently complementary to hybridize with a template. The term primer site refers to the area of the target DNA to which a primer hybridizes. The term primer pair means a set of primers including a 5′ upstream primer that hybridizes with the 5′ end of the DNA sequence to be amplified and a 3′, downstream primer that hybridizes with the complement of the 3′ end of the sequence to be amplified.

The term “probe” or “hybridization probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified by hybridization. “Probes” or “hybridization probes” are nucleic acids capable of binding in a base-specific manner to a complementary strand of nucleic acid. Such probes include peptide nucleic acids, as described in Nielsen et al. (1991. Science 254:1497-1500). Hybridizations are usually performed under “stringent conditions”, for example, at a salt concentration of no more than 1M and a temperature of at least 25° C. For example, conditions of 5×SSPE 750 mM NaCl, 50 mM NaPhosphate, 5 mM EDTA, pH 7.4) and a temperature of 25° C. to 30° C. are suitable for allele-specific probe hybridizations. Although this particular buffer composition is offered as an example, one skilled in the art could easily substitute other compositions of equal suitability.

The term “sequencing” as used herein means a process for determining the order of nucleotides in a nucleic acid. A variety of methods for sequencing nucleic acids are well known in the art. Such sequencing methods include the Sanger method of dideoxy-mediated chain termination as described, for example, in Sanger et al. 1977. Proc Natl Acad Sci 74:5463, which is incorporated herein by reference (see, also, “DNA Sequencing” in Sambrook et al. (eds.), Molecular Cloning: A Laboratory Manual (Second Edition), Plainview, N.Y.: Cold Spring Harbor Laboratory Press (1989), which is incorporated herein by reference). A variety of polymerases including the Klenow fragment of E. coli DNA polymerase I; Sequenase™ (T7 DNA polymerase); Taq DNA polymerase and Amplitaq can be used in enzymatic sequencing methods. Well known sequencing methods also include Maxam-Gilbert chemical degradation of DNA (see Maxam and Gilbert, Methods Enzymol. 65:499 (1980)), which is incorporated herein by reference, and “DNA Sequencing” in Sambrook et al., supra, 1989). One skilled in the art recognizes that sequencing is now often performed with the aid of automated methods.

The term “schizophrenia” refers to its conventional meaning, e.g., a mental disorder diagnosed according to the Research Diagnostic Criteria (RDC) (Spitzer R L et al. 1978. Arch Gen Psychiatry 35:773-782) and the Diagnostic and Statistical Manual of Mental Disorders, Fourth Edition (DSM-IV) (American Psychiatric Association 1994) using a best estimate consensus procedure (Baron M. et al, 1994. Psychiatr Genet 4:43-55). The term further contemplates schizophrenia related disorders, as described herein below.

The terms “trait” and “phenotype” are used interchangeably herein and refer to any visible, detectable or otherwise measurable property of an organism such as symptoms of, or susceptibility to a disease or a disorder, specifically schizophrenia.

### Polymorphism of the Invention

The present invention discloses genes and polymorphism associated with schizophrenia or predisposition to schizophrenia. In particular, the present invention discloses the linkage of Abelson Helper Integration Site 1 (AHI1) gene to schizophrenia susceptibility. The present invention further discloses the linkage of a gene of unknown function, C6orf217, with schizophrenia. The polymorphism and gene linkage disclosed by the present invention are useful in the diagnosis of schizophrenia, and furthermore, can serve as a means for identifying new treatments of the disorder.

The invention is based in part on an autosomal scan of Arab Israeli families, conducted by inventors of the present invention and co-workers, showing a linkage of schizophrenia to chromosome 6q23 that was significant at a genomewide level with a non-parametric LOD score (NPL) of 4.60 (p=0.000004). A more refined linkage was then identified by typing additional 42 microsatellite markers on chromosome 6q between D6S1570 (91.3 Mb) and D6S281 (169.8 Mb) in the same sample (average inter-marker distance 1.6 Mb) (Levi et al, 2005 supra). Within this sample, the peak NPL rose to 4.98 (p=0.00000058) at D6S1626 (136.3 Mb), immediately adjacent to D6S292 (NPL 4.98, p=0.00000068), the marker that gave the highest NPL in the original genome scan (Lerer et al, 2003, supra). The putative susceptibility region (NPL-1) was reduced to 3.90 Mb; the peak multipoint parametric LOD score was 4.63 at D6S1626 and the LOD-1 interval was 2.10 Mb.

The present invention employs extensive genotyping of single nucleotide polymorphisms (SNPs) within and adjoining candidate genes located on the putative susceptibility region of chromosome 6q23 described above. The genotyping was conducted with samples obtained from the same families as were included in the genome scan, complemented by additional Arab Israeli nuclear families from the same geographic area.

For assay of genomic DNA, virtually any biological sample (other than pure red blood cells) is suitable. For example, convenient tissue samples include whole blood, semen, saliva, tears, urine, fecal material, sweat, buccal, skin and hair. For assay of cDNA or mRNA, the tissue sample must be obtained from an organ in which the target nucleic acid is expressed. According to certain embodiments, the genomic DNA sample is obtained from whole blood samples or EBV-transformed lymphoblast lines. The sample may be further processed before the detecting step. For example, the DNA in the cell or tissue sample may be separated from other components of the sample, may be amplified, etc. All samples obtained from a subject, including those subjected to any sort of further processing are considered to be obtained from the subject.

The present invention discloses for the first time the association of the AHI1 gene with schizophrenia. As described in details hereinbelow, SNPs associated with schizophrenia were identified using an automated method based on allele specific primer extension reaction. SNPs assayed were those located in the putative susceptibility region of chromosome 6q23. In a first amplification step the amplification primers hybridize to a site on target DNA generating an 80-200 bp amplicon, which includes the polymorphism. In a second extension reaction an extension primer is designed to anneal directly adjacent to the polymorphic site and to undergo allele specific extension. This extended primer gives rise to detectable products signifying the presence of a particular allelic form.

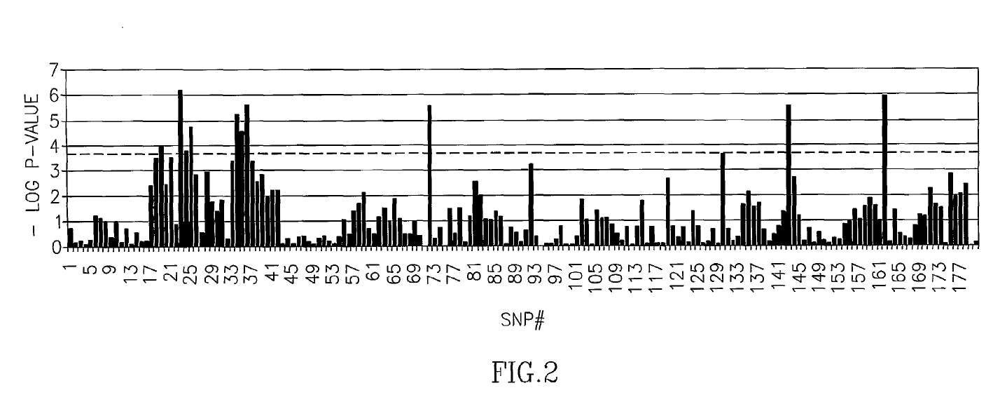

An area of ˜1 Mb area around the linkage peak at 136 Mb of the putative susceptibility region of chromosome 6q23 was assayed with a high density of SNPs. Single SNP association analysis of all 180 SNPs revealed a cluster of highly associated SNPs within the AHI1 gene at 135.7 Mb, which extends into the distal intergenic region between AHI1 and PDE7B (FIG. 2). Five known genes reside in this genomic region, namely MYB, AHI1, PDE7B, FAM54A and BCL2A, as well as a gene of unknown function, C6orf217, for which there is EST-cluster evidence. This gene is directly adjacent to AHI1.

The schizophrenia-associated SNPs according to the present invention are summarized in table 1 below.

TABLE 1
Schizophrenia-associated SNPs

| | | | | Over- |
|---|---|---|---|---|
| | | Position on | | transmitted |
| No. | rs Number | chromosome 6q | Gene | allele |
| 17 | rs6931735 | 135666504 | AHI1 | A |
| 18 | rs6912933 | 135669227 | AHI1 | A |
| 19 | rs9321501 | 135683110 | AHI1 | A |
| 20 | rs2746429 | 135714977 | AHI1 | T |
| 21 | rs2614258 | 135718895 | AHI1 | G |
| 23 | rs11154801 | 135781048 | AHI1 | C |
| 24 | rs7750586 | 135869366 | C6orf217 | A |
| 25 | rs9647635 | 135882749 | C6orf217 | A |
| 34 | rs7739635 | 136039471 | C6orf217 | C |
| 35 | rs9494332 | 136050301 | C6orf217 | G |
| 36 | rs1475069 | 136097927 | | A |
| 72 | rs797553 | 136613158 | FAM54A | G |
| 130 | rs642162 | 140256290 | | C |
| 143 | rs1414839 | 140934895 | | C |
| 162 | rs1239365 | 142615355 | | C |

Without wishing to be bound to a specific mechanism, the close proximity of AHI1 and the C6orf217 gene can indicate that the association with schizophrenia can be related to transcription regulation. The two genes share the same genomic region for their promoter sequences and therefore obligatorily affect each other's transcription regulation. It seems most likely that when one is transcribed the other is inhibited and vise versa. This could be more subtle if their regulation is different in specific tissues and then the interplay of the various transcription factors would be highly coordinated.

The mouse ashi1 gene was first identified as the integration site of helper provirus required for the Abelson murine leukemia retrovirus to replicate in vivo (Jiang X. et al. 2002. J Virol 76(18):9046-59). Recently, mutations in human AHI1 have been shown to cause the autosomal recessive brain disorder, the Joubert Syndrome (JS). The locus for JS had been mapped to chromosome 6q23 (Ferland R. J. et al. 2004. Nat Gene. 36(9):1008-13; Dixon-Salazar T. et al. 2004. Am J Hum Genet 75(6):979-87). JS is characterized by agenesis of the cerebellar vermis (the characteristic molar tooth malformation), ataxia, hypotonia, oculomotor apraxia, neonatal breathing abnormalities, and mental retardation. Additional phenotypic manifestations are diverse and may also include cerebral polymicrogyria and involvement of systems other than the central nerve system (CNS).

The primary transcript of the AHI1 gene is encoded by 28 exons and contains seven WD-40 repeats, a Src-homology 3 (SH3) domain and a coiled-coil domain in its N-terminal 140 amino acids (Jiang et al, 2002. supra; Close et al, 2004. BMC Genomics 5(1):33). Two alternate splice variants have been described. One is 3.7 Kb and includes an alternate exon “24a” in which the transcript results in a protein containing the WD-40 repeats, but missing the SH3 domain, suggesting that the two domains may function independently. Exon “27a” encodes an isoform that mirrors the full-length protein but lacks the far C-terminal sequence. The structure of the predicted protein encoded by AHI1 is particularly interesting in the context of the association with schizophrenia shown for the first time by the present invention. SH3 domains and WD-40 repeats are found in many signaling molecules and are known to mediate protein-protein interactions (Neer E. J. et al, 1994. Nature 371:297-300; Neer E. J. et al, 1996. Cell 84:175-178). The predicted structure of the AHI1 protein also contains nine putative SH-3 binding sites (Jiang et al, 2002. supra). Thus, AHI1 contains several putative motifs that have been shown to be present in signaling molecules and may play a role in signal transduction. Because of the high number of putative motifs mediating protein-protein interactions, it has been suggested that AHI1 may be a docking site or scaffold protein recruiting a number of other signaling molecules and modulating and integrating their action (Jiang et al. 2002, supra).

AHI1 mRNA is highly expressed in human fetal brain tissue and in the cerebellum and cerebral cortex of adult human brain (Ferland et al, 2004. supra). In developing mouse brain, ahi1 mRNA was detected at all time points analyzed. Maximal expression in cerebral cortex and cerebellum corresponded to the periods of maximal development of these two brain regions (Dixon-Salazar et al, 2004, supra). In mouse cerebellum the ahi1 expression pattern is predominantly midline and thus matches the cerebellar malformation associated with JS, suggesting that the protein may be a cell-autonomous modulator of axonal decussating (Ferland et al, 2004, supra).

The coiled-coil domain in the N-terminal of the predicted human AHI1 protein which contains 140 amino acids is entirely missing in the predicted proteins of both mouse and rat but is present in the predicted proteins of non-human primates and other mammals (Jiang et al, 2002. supra). The differences in the AHI1 sequence between species suggest that the portion of the gene encoding the N terminal segment might be particularly dynamic evolutionarily. Comparative genetic analysis of AHI1 suggested accelerated evolution of AHI1 in the human lineage, particularly the N terminal region of the gene, which was interpreted by Ferland et al, (2004. supra) as a consequence of directional selection.

The predicted structure and function of the protein encoded by AHI1, its expression pattern in brain and its putative evolutionary characteristics render AHI1 a highly relevant potential candidate gene involved in schizophrenia. Overt motor problems or other abnormalities characteristic of the JS phenotype were not observed in any of the families included in the analysis of the present invention, which are characterized by a high level of consanguinity. However, it is plausible that structural mutations less deleterious from the standpoint of gross brain development or variants that influence expression of the gene could influence brain development in utero and also post-natally in a more subtle way and thus contribute to susceptibility to schizophrenia. Brain imaging studies of schizophrenia have consistently identified increased volume of the lateral ventricles; slightly decreased overall brain; gray and white matter volumes decrease (between 2 and 3%) and regional volume decrease, in the hippocampus, thalamus and frontal lobes in patients with first-episode psychosis. The evidence for cerebellar abnormalities is equivocal. In childhood onset schizophrenia ventricular enlargement and gray matter reduction are also seen and are progressive, but temporal lobe changes are less prominent. Early postmortem findings reported neuronal disarray, especially in lamina II of the entorhinal cortex, and abnormal migration of subplate neurons in the neocortical white matter, suggesting prenatal neurodevelopmental abnormalities in neuronal migration and organization. More recent findings have emphasized abnormalities in neuronal size, arborization, and synaptic organization.

C6orf217 is primate-specific gene consisting of 10 exons and it has several alternatively splice isoforms. The predicted protein length depends on the splice isoform with a maximum of 135 amino acids with no similarity to any other known protein (Close J et al, 2004. BMC Genomics 5:33). Its largest open reading frame resides across exons one to three, while all the other exons seem to belong to the 3′ untranslated region (UTR). C6orf217 is expressed in brain, eye, kidney, testis, tongue, pancreas and lung during development as well as in the adult.

The present invention further discloses haplotypes associated with schizophrenia or predisposition to schizophrenia. Haplotype blocks were defined by performing 3-SNP-sliding window analysis across the entire 13.9 Mb genomic region using all 180 SNPs.

A total of 612 individual haplotypes were analyzed putting the Bonferroni cut-off value for significant haplotype association at 0.000082. A cluster of haplotypes, covering the AHI1 gene as well as the C6orf217 gene were shown to have a strong disease association. Haplotype association exceeded the association of single SNPs in this region in their degree of significance. Although SNP densities in these two gene regions were shown to be similar, the most significant associations, which withstand correction for multiple testing, were identified within the AHI1 gene.

In addition to the cluster region, two more individual haplotypes in other regions remain significant after correction for multiple testing. One is in an intergenic region at 138.1 Mb with a prevalence of 0.20 in the population and the other almost 1 Mb away at 139.0 Mb with a frequency of 0.14 and contributing SNPs residing in two different genes, namely C6orf63 and REPS1. The only region where the haplotype results support and strengthen the single SNP results is within the AHI1 gene.

The polymorphism of the present invention can be used in diagnostics tests, employing a variety of methodologies for the identification of individuals who are at increased risk of developing schizophrenia or suffers from schizophrenia.

Schizophrenia is one of a group of psychiatric conditions and disorders that exhibit a spectrum of similar phenotypes. Many of these conditions and disorders are found at increased frequency in family members of schizophrenic subjects, relative to their incidence in the general population. These factors make it likely that the same genetic mutations or alterations that contribute to schizophrenia susceptibility and/or pathogenesis are also involved in susceptibility to and/or pathogenesis of these conditions and disorders. Thus the methods and kits of the invention are also applicable to these related conditions and disorders.

Conditions related to schizophrenia include, but are not limited to: schizoaffective disorder, schizotypal personality disorder, schizotypy, a typical psychotic disorders, avoidant personality disorders, bipolar disorder, attention deficit hyperactivity disorder (ADHD), and obsessive compulsive disorder (OCD). Features and diagnostic criteria for these conditions are defined in the Diagnostic and Statistical Manual of Mental Disorders DSM-III, DSM III-R, DSM-IV, or DSM IV-R (American Psychiatric Association). As used herein, the term “schizophrenia” includes also “schizophrenia related conditions or disorders”. Thus, it is to be understood that the methods and kits disclosed by the present invention can also be used in a similar manner with respect to these conditions and disorders as described for schizophrenia itself.

According to one aspect, the present invention provides a method for diagnosing schizophrenia or predisposition to schizophrenia in a subject comprising (a) obtaining a sample comprising genetic material from the subject; (b) determining, in the genetic material, the nucleic acid sequence within a gene encoding AHI1 protein or a genomic region linked to the AHI1 gene; and (c) analyzing said nucleic acid sequence for polymorphism indicative of schizophrenia or a predisposition to schizophrenia.

According to another aspect, the present invention provides a method for diagnosing schizophrenia or predisposition to schizophrenia in a subject comprising (a) obtaining a sample comprising genetic material from the subject; (b) determining, in the genetic material, the nucleic acid sequence within a gene encoding AHI1 protein or a genomic region linked to the AHI1 gene; and (c) analyzing said nucleic acid sequence for the presence of at least one haplotype, wherein the presence of at least one AHI1 schizophrenia-associated haplotype is indicative of schizophrenia or predisposition to schizophrenia.

It is to be understood that “predisposition” or “susceptibility” to schizophrenia do not necessarily mean that the subject will develop schizophrenia but rather that the subject is, in a statistical sense, more likely to develop schizophrenia than an average member of the population. As used herein, “predisposition” or “susceptibility to schizophrenia may exist if the subject has one or more genetic determinants (e.g., polymorphic variants or alleles) that may, either alone or in combination with one or more other genetic determinants, contribute to an increased risk of developing schizophrenia in some or all subjects. Ascertaining whether a subject has any such genetic determinants according to the teaching of the present invention is useful, for example, for purposes of genetic counseling.

In general, if the polymorphism is located in a gene, it may be located in a noncoding or coding region of the gene. If located in a coding region the polymorphism can result in an amino acid alteration. Such alteration may or may not have an effect on the function or activity of the encoded polypeptide. When the polymorphism is located in a non-coding region it can cause alternative splicing, which again, may or may not have an effect on the encoded protein activity or function. It should be understood that diagnosing schizophrenia or predisposition to schizophrenia by detecting a variant gene product(s) are also encompassed within the scope of the present invention. As used herein a “variant gene product” refers to a gene product which is encoded by the variant allele comprising at lease one polymorphic sites according to the present invention, including, but not limited to, a full length gene product, an essentially full-length gene product and a biologically active fragment of the gene product. Biologically active fragments include any portion of the full-length polypeptide which confers a biological function on the variant gene product, including ligand binding and antibody binding. Ligand binding includes binding by nucleic acids, proteins or polypeptides, small biologically active molecules, or large cellular structures.

A variant gene product is also intended to mean gene products which have altered expression levels or expression patterns which are caused, for example, by the variant allele of a regulatory sequence(s). The term “regulatory sequence” is intended to include promoters, enhancers and other expression control elements (e.g., polyadenylation signals). Such regulatory sequences are described, for example, in Goeddel, Gene Expression Technology Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences).

The genetic material can be obtained from any suitable sample taken from the subject as described herein above. The subject can be an adult, child, fetus, or embryo. According to certain embodiments of the invention the sample is obtained prenatally, either from the fetus or embryo or from the mother (e.g., from fetal or embryonic cells that enter the maternal circulation). Typically, the sample obtained from the subject is processed before the detecting step, e.g. the DNA in the cell or tissue is separated from other components of the sample, and the target DNA is amplified as described herein below. All samples obtained from a subject, including those subjected to any sort of further processing, are considered to be obtained from the subject.

According to certain embodiments the diagnosis methods of the present invention are applied before the disease or condition manifests clinically. This may be advantageous for early intervention. Appropriate therapy may be administered to a susceptible subject (or to the subject's mother in the case of prenatal diagnosis) prior to development of the disease (e.g., prior to birth in the case of prenatal diagnosis). Since schizophrenia may be at least in part a developmental disorder, such early intervention may prove to be critical for prevention of the disease.

Detection of polymorphism in the target DNA typically requires amplification of DNA from the target samples. Methods for DNA amplification are known to a person skilled in the art. Most commonly used method for DNA amplification is PCR (polymerase chain reaction; see, for example, PCR Basics: from background to Bench, Springer Verlag, 2000; Eckert et al., 1991. PCR Methods and Applications 1:17). Additional suitable amplification methods include the ligase chain reaction (LCR), transcription amplification and self-sustained sequence replication, and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

Various tools for the detection of polymorphism on a target DNA are known in the art, including, but not limited to, allele-specific probes, allele specific primers, direct sequencing, denaturing gradient gel electrophoresis and single-strand conformation polymorphism. Preferred techniques for SNP genotyping should allow large scale, automated analysis, which do not require extensive optimization for each SNP analyzed.

The phrase “detecting a polymorphism” or “detecting a polymorphic variant” as used herein generally refers to determining which of two or more polymorphic variants exists at a polymorphic site. For purposes of description, if a subject has any sequence other than a defined reference (e.g. the sequence present in the human draft genome) at a polymorphic site, the subject may be said to exhibit the polymorphism. In general, for a given polymorphism, any individual will exhibit either one or two possible variants at the polymorphic site (one on each chromosome). This may, however, not be the case if the individual exhibits one more chromosomal abnormality such as deletions.

Detection of a polymorphism or polymorphic variant in a subject (genotyping) may be performed by sequencing, similarly to the manner in which the existence of a polymorphism is initially established. However, once the existence of a polymorphism is established a variety of more efficient methods may be employed. Many such methods are based on the design of oligonucleotide probes or primers that facilitate distinguishing between two or more polymorphic variants.

Oligonucleotides that exhibit differential or selective binding to polymorphic sites may readily be designed by one of ordinary skill in the art. For example, an oligonucleotide that is perfectly complementary to a sequence that encompasses a polymorphic site (i.e., a sequence that includes the polymorphic site within it or at least at one end) will generally hybridize preferentially to a nucleic acid comprising that sequence as opposed to a nucleic acid comprising an alternate polymorphic variant.

The design and use of allele-specific probes for analyzing polymorphisms is described, for example, in U.S. Pat. No. 5,348,855 and International Application WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Typically, a probe comprises a region of nucleotide sequence that hybridizes to at least about 10, preferably to about 10 to 15, more preferably to about 20-25 and most preferably to about 25-35 consecutive nucleotides of a nucleic acid molecule. Preferably, the probes are designed as to be sufficiently specific to be able to discriminate the targeted sequence for only one nucleotide variation. According to certain embodiments, the probes are labeled or immobilized on a solid support by any suitable method as is known to a person skilled in the art. The probes can be used in Southern hybridization to genomic DNA or Northern hybridization to mRNA; the probes can also be used to detect PCR amplification products. By assaying the hybridization to an allele specific probe, one can detect the presence or absence of a polymorphism in a given sample. Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence. High-Throughput parallel hybridizations in array format are particularly preferred to enable simultaneous analysis of a large number of samples.

Alternative method for the detection of polymorphism on a target DNA utilizes allele-specific primers, as described herein above. The direct analysis of the sequence of polymorphisms of the present invention can be accomplished using either the dideoxy chain termination method or the Maxam Gilbert method (see Sambrook et al., 1989. supra; Zyskind et al, Recombinant DNA Laboratory Manual, Acad. Press, 1988). It should be recognized that the field of DNA sequencing has advanced considerably in the past several years, specifically in reliable methods of automated DNA sequencing and analysis. These advances and those to come are explicitly encompassed within the scope of the present invention. As is known to a person skilled in the art, an amplified product can be sequenced directly or subcloned into a vector prior to sequence analysis.

Alleles of target sequences can be differentiated using single-strand conformation polymorphism analysis, which identifies base differences by alteration in electrophoretic migration of single stranded PCR products. Amplified PCR products can be generated as described above, and heated or otherwise denatured, to form single stranded amplification products. Single-stranded nucleic acids may refold or form secondary structures which are partially dependent on the base sequence. The different electrophoretic mobility of single-stranded amplification products can be related to base-sequence difference between alleles of target sequences.

Another method for rapid and efficient SNP analysis makes use of thermal denaturation differences due to differences in DNA base composition. In one embodiment of this test, allele specific primers are designed as above to detect biallelic SNP with the exception that to one primer a 5′ GC tail of 26 bases is added. After PCR amplification with a single, common reverse primer, a fluorescent dye that binds preferentially to dsDNA (e.g., SYBR Green 1) is added to the tube and then the thermal denaturation profile of the dsDNA product of the PCR amplification is determined. Samples homozygous for the SNP amplified by the GC tailed primer will denature at the high end of the temperature scale, while samples homozygous for the SNP amplified by the non-GC tagged primer will denature at the low end of the temperature scale. Heterozygous samples will show two peaks in the thermal denaturation profile.

The invention further contemplates modifications of the methods described above, including, but not limited to allele-specific hybridization on filters, allele-specific PCR, fluorescence allele-specific PCR, PCR plus restriction enzyme digest (RFLP-PCR), denaturing capillary electrophoresis, dynamic allele-specific hybridization (DASH), 5′ nuclease (Taq-Man™) assay, and the primer extension and time-of-flight mass spectrometry. According to certain currently preferred embodiments, the polymorphism of the present invention is detected using the primer extension and time-of-flight mass spectrometry method as exemplified herein below.

The diagnosis of a nucleic acid samples obtained from a subject to be assessed for schizophrenia or predisposition to schizophrenia, by any of the methods described above, can be based on the presence of a singles polymorphism or on a group of polymorphism. According to certain embodiments, the polymorphism site has a reference sequence number selected from the group consisting of rs6931735, rs6912933, rs9321501, rs2746429, rs2614258, rs11154801, rs7750586, rs9647635, rs7739635, rs9494332, rs1475069, rs797553, rs642162, rs1414839 and rs1239365 on chromosome 6q23.

According to other embodiments, the presence of at least one of Adenine (A) at any one of the polymorphic sites rs6931735, rs6912933, rs9321501, rs7750586, rs9647635 or rs1475069; Thymine (T) at the polymorphic site rs2746429; Guanine (G) at any one of the polymorphic sites rs2614258, rs9494332 or 797553; or Cytosine (C) at any one of the polymorphic sites rs1154801, rs7739635; rs642162; rs1414839 or rs1239365 indicates that the subject has schizophrenia or predisposition to schizophrenia.

According to yet further embodiments, schizophrenia or predisposition to schizophrenia is diagnosed by the presence of at least one haplotype, selected from the group consisting of a haplotype comprising the nucleotides TGG at rs878175, rs6931735, and rs6912933 respectively; a haplotype comprising the nucleotides AAA at rs6931735, rs6912933, and rs9321501 respectively; a haplotype comprising the nucleotides GGC at rs6931735, rs6912933, and rs9321501 respectively; a haplotype comprising the nucleotides AAT at rs6912933, rs9321501 and rs2746429 respectively; a haplotype comprising the nucleotides GCC at rs6912933, rs9321501, rs2746429, respectively; a haplotype comprising the nucleotides ATG at rs9321501, rs2746429, and rs2614258, respectively; a haplotype comprising the nucleotides CCA at rs9321501, rs2746429, and rs2614258, respectively; a haplotype comprising the nucleotides ATA at rs667520, rs999638 and rs683122, respectively; a haplotype comprising the nucleotides TAA at rs2876391, rs1188852, and rs1188863, respectively or any combination thereof.

The diagnostic methods of the present invention are extremely valuable as they can, in certain circumstances, be used to initiate preventive treatments or to allow an individual carrying a significant haplotype to foresee warning signs such as minor symptoms. The knowledge of a potential predisposition, even if this predisposition is not absolute, might contribute in a very significant manner to treatment efficacy.

The means and methods of the present invention are used to determine whether or not an individual has a polymorphism located on chromosome 6q23 within AHI1 gene or a region linked to the AHI1 gene, shown for the first time by the present invention to be associated with schizophrenia. Population studies that compare the frequency of this polymorphism in the general population and the frequency of the polymorphism in persons with schizophrenia show that this polymorphism is a genetic risk factor to have or develop schizophrenia. The information disclosed herein regarding the polymorphism can be used either as a prognosis tool to identify individuals with increased risk for developing schizophrenia at a future point in time, or as a diagnostic tool to identify individuals suspect to have schizophrenia by a clinical exam who may therefore be diagnosed as being more likely to have schizophrenia, or other related diseases and disorders such as schizoaffective disorder-bipolar, schizoaffective disorder-depression, schizotypal personality disorder, non-affective psychotic disorder (e.g. schizophreniform disorder, delusional disorder, psychotic disorder not otherwise specified (NOS)), or mood-incongruent psychotic depressive disorder or paranoid or schizoid personality disorder.

The following examples are presented in order to more fully illustrate the preferred embodiments of the invention. They should in no way be construed, however, as limiting the broad scope of the invention.

### EXAMPLES

### Materials and Methods

### Family Sample and Diagnostic Methods

Families of Arab Israeli origin with two or more members affected with schizophrenia were systematically recruited from the catchments area of the Taibe Regional Mental Health Center (Lerer et al, 2003. supra; Levi et al, 2005. supra). This clinic serves a population of ˜66,000 people living in adjacent Arab Israeli towns and villages in the central region of Israel. The project was approved by the Helsinki Committee (Internal Review Board) of the Hadassah—Hebrew University Medical Center and written informed consent was obtained from all subjects. The Arab Israeli population is an ethnically homogeneous group that has a high birthrate, an unusually high level of consanguinity (˜25% first cousin marriages) and a low rate of intermarriage with other population groups in Israel. The sample was primarily derived from three Arab Israeli towns that were founded approximately 200-250 years ago by a limited number of families. In subsequent years there was immigration into the towns but the major population increase has been due to a high birthrate and low infant mortality in the past 75 years. Traditionally, marriages are within the community, often within the same extended patrilineal clan.

One nuclear family was selected from each large family included in the basic genome scan or recruited subsequently. The criteria for selecting a family were largest number of affected offspring and at least one parent, preferably both, recruited. The sample that was available for family-based association studies included 53 families (including the families recruited subsequent to the genome scan) with 190 individuals that provided DNA samples, of whom 85 were affected. Of the 53 families, 34 families were “triad” families including affected proband plus both parents; and 19 families had 2 or more affected offspring (10 with 2 affected, 6 with 3 affected, 2 with 4 affected and 1 with 5 affected). Of these 19 families, 10 have both parents recruited and 9 have one parent recruited (plus one or more unaffected sibling).

To establish psychiatric diagnosis, subjects were interviewed with the Schedule for Affective Disorders and Schizophrenia-Lifetime Version (SADS-L) (Spitzer R and Endicott J eds. 1977. The schedule for affective disorders and schizophrenia, lifetime version. 3rd edition. New York State Psychiatric Institute, New York) and were questioned about psychiatric symptoms in the family according to the Family History Research Diagnostic Criteria (FH-RDC) (Andreasen N C et al, 1977. Arch Gen Psychiatry 34:1229-1235). Medical records of hospitalizations and clinic care were obtained for affected individuals. The completed SADS-L interview form, FH-RDC information and medical records were reviewed by two experienced members of the research team and, in cases where consensus was not achieved, by the principal investigator. Lifetime diagnoses were established according to the Research Diagnostic Criteria (RDC) (Spitzer R L et al, 1978. Arch Gen Psychiatry 35:773-782) and the Diagnostic and Statistical Manual of Mental Disorders, Fourth Edition (DSM-IV) (American Psychiatric Association 1994) using a best estimate consensus procedure (Baron M et al, 1994. Psychiatr. Genet 4:43-55). All diagnostic evaluations were completed without knowledge of the genotyping data. For the genome scan and fine mapping, three diagnostic categories were employed—broad, core and narrow (Lerer et al, 2003, supra; Levi et al, 2005. supra). For the SNPs identification according to the teaching of the present invention, only the broad category was employed because this category consistently yielded the strongest results in the genome scan and in the subsequent fine mapping, and also in order to reduce the extent of correction for multiple testing. In the current sample this category encompassed 65 subjects affected with schizophrenia (44 probands and 21 affected siblings) according to RDC; 17 subjects affected with schizoaffective disorder (9 probands and 8 affected siblings) and 3 siblings affected with unspecified functional psychosis. Other diagnoses potentially included in the broad diagnostic category are not in fact represented in the sample.

### Genotyping

Information about SNPs was obtained from public databases (Ensembl, NCBI) as well as from the Celera database. Only validated SNPs with a minor allele frequency >0.2 in Caucasians were considered for genotyping. The Sequenom MassARRAY platform (Sequenom, San Diego Calif.) is based on an allele specific primer extension reaction, which is detected by MALDI-TOF MS (matrix assisted laser desorption ionization time of flight mass spectrometry) technology. The protocol for high multiplex homogeneous MassEXTEND (hME) reactions (Sequenom, San Diego Calif., application notes) was used, with a variation of the recommended amount of 2 ng DNA per reaction to from 0.5 ng to 4 ng DNA per reaction, depending on the assay. Genotyping assays were designed as multiplex reactions using SpectroDESIGNER software version 2.0.7 (Sequenom, San Diego Calif.) after verifying that the SNPs do not reside in repetitive elements. The acquired genotypes were checked for deviations from Mendelian inheritance using the program PedManager (http://www.broad.mit.edu/ftp/distribution/software/pedmanager). Ambiguous markers were removed from the analysis in any specific family, if re-examination of the raw data did not resolve the ambiguity. For removal of a specific family from analysis the genotypes were set to zero (unknown genotype) for all family members at a given locus. The rate of genotyping errors was below 2% for all markers in the entire sample. 30% of the SNPs selected in the course of the present assays were not polymorphic in the Arab Israeli sample described hereinabove.

### Statistical Analysis

To check for association between a single SNP or a haplotype composed of more than one SNP and the hypothetical disease locus, PBAT Version 3.0 was used (Lange C. et al, 2004. Am J Hum Genet 74(2):367-369; Steen K. V. et al., 2005. Hum Genomics. 2(1):67-69). The PBAT software incorporates an extended and improved transmission disequilibrium test (TDT) (Spielman R. S. et al, 1993. Am J Hum Genet 52:506-516) based on a linear combination of offspring genotypes and traits. All analysis was done within a linkage interval (Lerer et al, 2003. supra; Levi et al, 2005. supra); therefore the PBAT statistic was calculated under the null hypothesis of linkage and no association using the sandwich option (sw) for robust estimation of the variance, conditioning on traits and parental genotypes (FBAT/PBAT User Manual, Carroll et al, 1998). Haplotype analysis was restricted to adjacent SNPs; because the algorithm assumes no recombination, three separate input files were created, omitting known recombination hotspots. The mode of inheritance of schizophrenia is complex and therefore the additive mode was used, as suggested by the manual when the exact mode of inheritance is unknown. The minimal number of informative families was limited to 10 and the minimal haplotype frequency cut-off was set to 0.05.

Stringent Bonferroni correction was used in order to correct for multiple testing. In cases were there is high LD between SNPs this method might be overly conservative. The correction was done separately for single SNP and haplotype analysis.

Haploview Version 3.2 (http://www.broad.mit.edu/mpg/haploview) was used to calculate intermarker LD between all SNP pairs within a 1 Mb interval and to generate a graphical view of the LD pattern across the entire genomic region. Haplotype blocks were defined using the confidence interval algorithm (Gabriel S. B. et al, 2002. Science 21:296(5576):2225-2229) implemented by Haploview.

### Example 1

### Schizophrenia-Associated SNPs

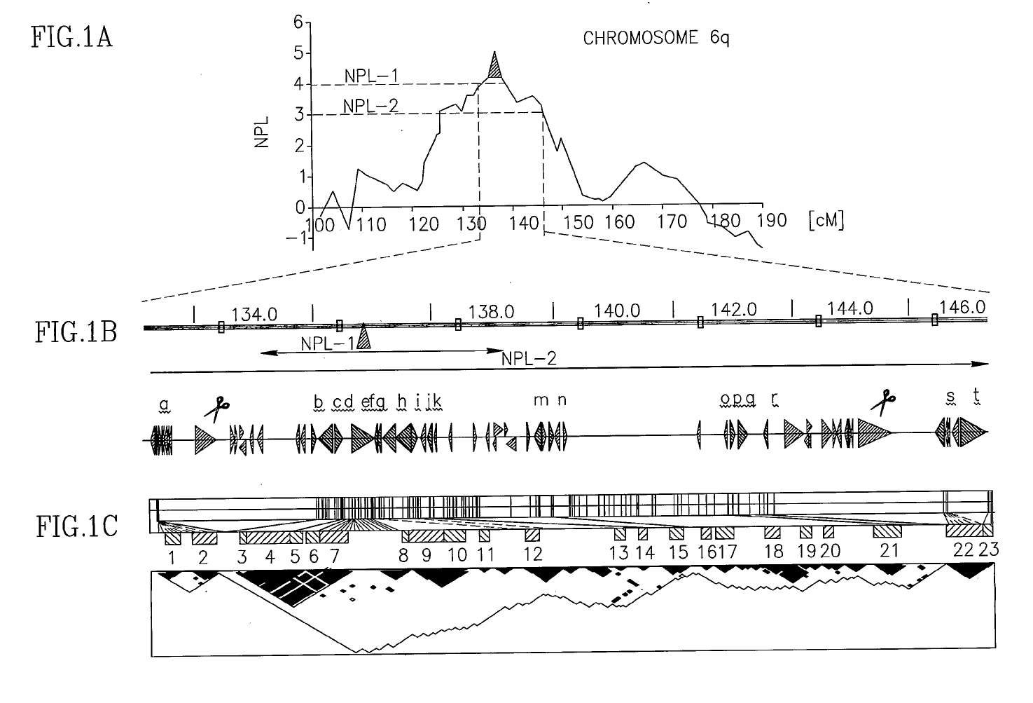

Fifty-three families from a sample that showed linkage to a schizophrenia susceptibility locus on chromosome 6q23 (FIG. 1A) were examined, by genotyping 219 SNPs spanning a genomic region of 13.9 Mb within the NPL-2 confidence interval of the linkage peak (Lerer et al, 2003. supra; Levi et al, 2005. supra). SNPs with MAF <0.05 (n=15), with less than 10 informative families (n=20, randomly distributed across the entire interval) or showing deviation from Hardy-Weinberg equilibrium (HWE) among the parents (n=4) were excluded from the analysis, leaving a total of 180 SNPs. The region harbors 69 known genes with the majority showing brain expression (FIG. 1B). SNP density was highest in a 1 Mb area around the linkage peak at 136 Mb with a total of 58 SNPs at an average inter-SNP distance of 17.0 kb (FIG. 1C); the average SNP density of the remaining SNPs was 66.6 kb. The LD pattern of the region is shown in FIG. 1D.

Single SNP association analysis of all 180 SNPs revealed a cluster of highly associated SNPs within the AHI1 gene at 135.7 Mb, which extends into the distal intergenic region between AHI1 and PDE7B (FIG. 2). Five known genes reside in this ˜1 Mb genomic region, namely MYB, AHI1, PDE7B, FAM54A and BCL2A, as well as a gene of unknown function, C6orf217, for which there is EST-cluster evidence. This gene is directly adjacent to AHI1. After conservative Bonferroni correction for multiple testing (180 tests, Bonferroni cut-off p-value 0.00028), two out of the six SNPs within AHI1, namely rs9321501 (p=0.00010) and rs11154801 (p=0.00000071), remain significant while two additional SNPs within AHI1 almost meet the cut-off criterion (rs6912933, p=0.00032 and rs2614258, p=0.00032). Within the C6orf217 gene four SNPs withstand correction, two SNPs at the 5′-end, rs7750586 (p=0.00017) and rs9647635 (p=0.000019), and two SNPs at the 3′-end, rs7739635 (p=0.0000059) and rs9494332 (0.000030). The last significant SNP in this cluster (rs1475069, p=0.0000025) is located ˜48 kb distal to the last annotated exon of C6orf217 in an intergenic region between it and PDE7B.

### Example 2

### Schizophrenia-Associated Haplotypes

In order to check whether applying the stringent Bonferroni correction lead us to miss other SNPs in LD with the trait, the threshold was relaxed to 0.0056 (i.e. 20-fold). Sixteen SNPs were added, ten of which were mapped in or around the AHI1 high LD cluster. The other six were scattered throughout the remaining interval.

The AHI1 gene and the C6orf217 gene are located head to head with only 55 bp distances between the 5′ ends of the two genes. Therefore their promoters obligatorily share the same genomic region, since promoters extend between −45 bp to −1000 bp from the transcription start sites. The two genes share a transcription factor binding-site (TFBS) for CREB (−30 bp for AHI1 and −12 bp for the non-annotated gene). Moreover TFBSs for AHI1 gene are located within the non-annotated gene including, for example, RFX1 (−64 bp), STAT1 (−272 bp), NF-AT (−385 bp), API (−682 bp) and AML-1a (−992 bp).

The SNPs in the genomic region spanning from MYB to C6orf217 are in high LD with each other and therefore observed associations in this region are not independent. Single SNP analysis in this ˜0.5 Mb region is not likely to further refine the location of disease association. In addition to the cluster of associated SNPs within the high LD region from MYB to C6orf217 four additional SNPs in different genomic locations that withstand Bonferroni correction were identified. The first of these, rs797553 (p=0.0000030), is located in the 5′ UTR of the FAM54A gene at 136.6 Mb; next are two intergenic SNPs which are located in a genomic region with no identified genes, spanning from approximately 140-142 Mb (rs642162, p=0.00024; rs1414839, p=0.0000029). The last SNP that showed a significant association to schizophrenia is rs1239365 (p=0.0000012) at 142.6 Mb.

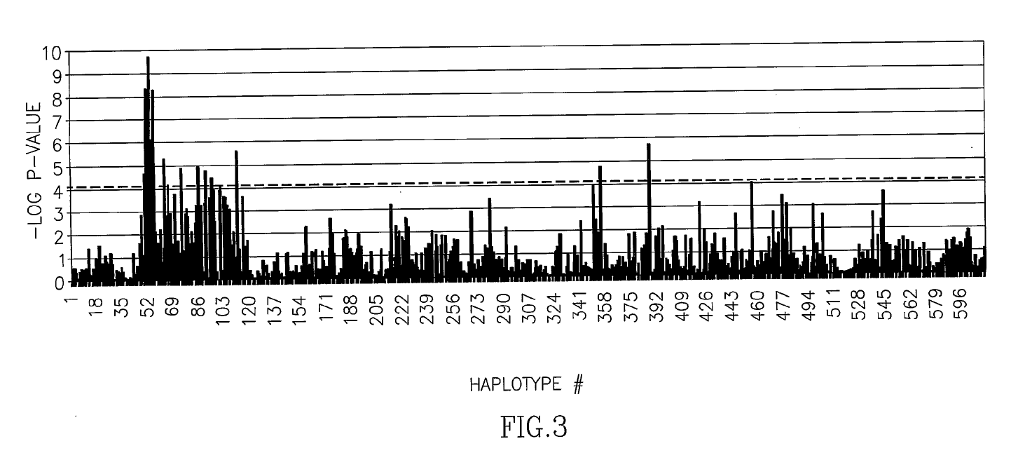

In order to further explore the genomic region and to increase the information provided by single SNP analysis 3-SNP-sliding window analysis across the entire 13.9 Mb genomic region was performed, using all 180 SNPs. A total of 612 individual haplotypes were analyzed putting the Bonferroni cut-off value for significant haplotype association at 0.000082. A summary of the haplotype results is shown in FIG. 3. There is a cluster of haplotypes showing strong disease association covering the AHI1 gene as well as C6orf217 (haplotypes #50-112), with haplotype association exceeding the association of single SNPs in this region in their degree of significance. SNP densities in these two gene regions being similar, it is noteworthy that the most significant associations, which withstand correction for multiple testing, are within the AHI1 gene. Three consecutive haplotype windows form the peak of the cluster composed of SNPs rs6931735, rs6912933, rs9321501, rs2746429 and rs2614258, with rs9321501 being part of all three haplotype windows. With only two common haplotypes in this region, the most significant association was observed with the frequent AAT (2.2.2.) haplotype (frequency: 0.67) consisting of SNPs rs6912933, rs9321501 and rs2746429 (p=0.00000000017) and a similar, but less significant trend, with the complementary GCC (1.1.1) haplotype (frequency: 0.22, p=0.0000019). Table 2 presents the individual haplotype compositions of the significant cluster.

TABLE 2
Schizophrenia-associated haplotypes

| | | | Haplo- | |
|---|---|---|---|---|
| SNP 1 | SNP 2 | SNP 3 | type | Gene |
| rs878175 | rs6931735 | rs6912933 | TGG | intergenic. AHI1. AHI1 |
| rs6931735 | rs6912933 | rs9321501 | AAA | AHI1 |
| rs6931735 | rs6912933 | rs9321501 | GGC | AHI1 |
| rs6912933 | rs9321501 | rs2746429 | AAT | AHI1 |
| rs6912933 | rs9321501 | rs2746429 | GCC | AHI1 |
| rs9321501 | rs2746429 | rs2614258 | ATG | AHI1 |
| rs9321501 | rs2746429 | rs2614258 | CCA | AHI1 |
| rs667520 | rs999638 | rs683122 | ATA | Intergenic |
| rs2876391 | rs1188852 | rs1188863 | TAA | C6orf63.intergenic.REPS1 |

In addition to the cluster region, two more individual haplotypes in other regions that remain significant after correction for multiple testing were found. One is in an intergenic region at 138.1 Mb with a prevalence of 0.20 in the population and the other almost 1 Mb away at 139.0 Mb with a frequency of 0.14 and contributing SNPs residing in two different genes namely C6orf63 and REPS1. The only region where the haplotype results support and strengthen the single SNP results is within the AHI1 gene. Bearing in mind that even with the Bonferroni correction for multiple testing at a significance level of 0.05 there is a 5% chance of false positive findings, the AHI1 gene is the most consistent location of association. However, due to the lower SNP density in other regions, the possibility that chromosome 6q23 harbors more than one gene associated with schizophrenia cannot be definitively excluded.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1.-39. (canceled)

40. A method for diagnosing schizophrenia or predisposition to schizophrenia in a subject comprising:
obtaining a sample comprising genetic material from the subject;
determining, in the genetic material, of (a) the nucleic acid sequence within the AHI1 gene or a genomic region linked to the AHI1 gene comprising a gene designated C6orf217; or (b) the nucleic acid sequence within a genomic region between a gene designated C6orf217 and the PDE7B gene; or (c) the nucleic acid sequence within a gene encoding FAM54A protein; or (d) the nucleic acid sequence at about 140-142 Mb on chromosome 6q23; and
analyzing said nucleic acid sequence for polymorphism indicative of schizophrenia or a predisposition to schizophrenia.

41. The method of claim 40, wherein the determining of the nucleic acid sequence is within the AHI1 gene or a genomic region linked to the AHI1 gene comprising a gene designated C6 orf217, and the analyzing the nucleic acid sequence for polymorphism comprises determining the identity of at least one polymorphic site within the AHI1 gene having a reference sequence number on chromosome 6q23 selected from the group consisting of rs6931735, rs6912933, rs9321501, rs2746429, rs2614258 and rs11154801.

42. The method of claim 41, wherein the presence of Adenine at rs6931735, rs6912933 or rs9321501; Thymine at rs2746429; Guanine at rs2614258 or Cytosine at rs11154801, indicates that the subject has schizophrenia or predisposition to schizophrenia.

43. The method of claim 40, wherein the determining of the nucleic acid sequence is within the AHI1 gene or a genomic region linked to the AHI1 gene comprising a gene designated C6 orf217, and the analyzing the nucleic acid sequence for polymorphism comprises determining the identity of at least one polymorphic site within the C6orf217 gene having a reference sequence number on chromosome 6q23 selected from the group consisting of rs7750586, rs9647635, rs7739635 and rs9494332.

44. The method of claim 43, wherein the presence of Adenine at rs7750586 or rs9647635; Cytosine at rs7739635; or Guanine at rs9494332 indicates that the subject has schizophrenia or predisposition to schizophrenia.

45. The method of claim 40, wherein the determining of the nucleic acid sequence within the genomic region is between the gene designated C6 orf217 and the PDE7B gene, and the analyzing the nucleic acid sequence for polymorphism comprises determining the identity of a polymorphic site having a reference sequence number rs1475069 on chromosome 6q23.

46. The method of claim 45 wherein the presence of Adenine at rs1475069 indicates that the subject has schizophrenia or predisposition to schizophrenia.

47. The method of claim 40, wherein the determining of the nucleic acid sequence is within a gene encoding FAM54A protein, and the analyzing of the nucleic acid sequence for polymorphism comprises determining the identity of a polymorphic site having a reference sequence number rs797553 on chromosome 6q23.

48. The method of claim 47 wherein the presence of Guanine at rs797553 indicates that the subject has schizophrenia or predisposition to schizophrenia.

49. The method of claim 40, wherein the determining is of the nucleic acid sequence at about 140-142 Mb on chromosome 6q23, and the analyzing of the nucleic acid sequence for polymorphism comprises determining the identity of at least one polymorphic site having a reference sequence number selected from the group consisting of rs642162, rs1414839 and rs1239365 on chromosome 6q23.

50. The method of claim 49, wherein the presence of Cytosine at rs642162; rs1414839; or rs1239365 indicates that the subject has schizophrenia or predisposition to schizophrenia.

51. The method of claim 49, wherein analyzing said nucleic acid sequence for polymorphism comprises determining the presence of at least one haplotype; wherein the presence of at least one schizophrenia-associated haplotype is indicative of schizophrenia or predisposition to schizophrenia.

52. The method of claim 51 wherein the schizophrenia-associated haplotype comprises at least two polymorphic sites having reference sequence number selected from the group consisting of rs878175, rs6931735, rs6912933, rs9321501, rs2746429 and rs2614258 on chromosome 6q23.

53. The method of claim 52, wherein the schizophrenia-associated haplotype comprises polymorphic sites having reference sequence number rs878175, rs6931735 and rs6912933 on chromosome 6q23.

54. The method of claim 53, wherein the nucleotide identity at the reference sequence numbers rs878175, rs6931735 and rs6912933 is TGG respectively.

55. The method of claim 52, wherein the schizophrenia-associated haplotype comprises polymorphic sites having reference sequence number rs6931735, rs6912933, and rs9321501 on chromosome 6q23.

56. The method of claim 55, wherein the nucleotide identity at the reference sequence numbers rs6931735, rs6912933, and rs9321501, respectively, is selected from the group consisting of AAA and GGC.

57. The method of claim 52, wherein the schizophrenia-associated haplotype comprises polymorphic sites having reference sequence number rs6912933, rs9321501 and rs2746429 on chromosome 6q23.

58. The method of claim 57, wherein the nucleotide identity at the reference sequence numbers rs6912933, rs9321501 and rs2746429, respectively, is selected from the group consisting of AAT and GCC.

59. The method of claim 52, wherein the schizophrenia-associated haplotype comprises polymorphic sites having a reference sequence number rs9321501, rs2746429 and rs2614258 on chromosome 6q23.

60. The method of claim 59, wherein the nucleotide identity at the reference sequence numbers rs9321501, rs2746429 and rs2614258, respectively, is selected from the group consisting of ATG and CCA.

61. A method for diagnosing schizophrenia or predisposition to schizophrenia in a subject comprising:
obtaining a sample comprising genetic material from the subject; and
determining, in the genetic material, (a) the nucleic acid sequence within a genomic region between the IL22RA2 gene and the TNFAIP3 gene at about 138.1 Mb on chromosome 6q23; or (b) the identity of the nucleic acid sequence within the C6orf55 gene and the REPS1 gene at about 139 Mb on chromosome 6q23; and
analyzing said nucleic acid sequence for the presence of at least one haplotype;
wherein the presence of at least one schizophrenia-associated haplotype is indicative of schizophrenia or predisposition to schizophrenia.

62. The method of claim 61, wherein the determining of the nucleic acid sequence is within a genomic region between the IL22RA2 gene and the TNFAIP3 gene at about 138.1 Mb on chromosome 6q23 and the schizophrenia-associated haplotype comprises at least two polymorphic sites having reference sequence number selected from the group consisting of rs667520, rs999638 and rs683122 on chromosome 6q23.

63. The method of claim 62, wherein the schizophrenia-associated haplotype comprises polymorphic sites having reference sequence number rs667520, rs999638 and rs683122 on chromosome 6q23.

64. The method of claim 63, wherein the nucleotide identity at the reference sequence numbers rs667520, rs999638 and rs683122 is ATA respectively.

65. The method of claim 61, wherein the determining the identity of the nucleic acid sequence is within the C6orf55 gene and the REPS1 gene at about 139 Mb on chromosome 6q23; and the schizophrenia-associated haplotype comprises at least two polymorphic sites having reference sequence number selected from the group consisting of rs2876391, rs1188852 and rs1188863 on chromosome 6q23.

66. The method of claim 65, wherein the schizophrenia-associated haplotype comprises polymorphic sites having reference sequence number rs2876391, rs1188852 and rs1188863 on chromosome 6q23.

67. The method according to claim 66, wherein the nucleotide identity at the reference sequence numbers rs2876391, rs1188852 and rs1188863 respectively, is TAA.

68. An isolated polynucleotide designed to specifically detect a naturally occurring polymorphic variant of a polymorphism indicative of schizophrenia or predisposition to schizophrenia within a region on chromosome 6q23 from about 136 Mb to about 142 Mb.

69. The isolated polynucleotide of claim 68, wherein the polymorphism indicative of schizophrenia or predisposition to schizophrenia comprises at least one polymorphic site having a reference sequence number selected from the group consisting of rs6931735, rs6912933, rs9321501, rs2746429, rs2614258, rs11154801, rs7750586, rs9647635, rs7739635, rs9494332, rs1475069 rs797553, rs642162, rs1414839 and rs1239365 on chromosome 6q23.

70. The isolated polynucleotide of claim 69, comprising from about 10 to about 100 contiguous nucleotides and designed to specifically hybridize to a nucleic acid molecule comprising at least one polymorphic site of the polymorphism indicative of schizophrenia or predisposition to schizophrenia.

71. The isolated polynucleotide of claim 70, comprising from about 15 to about 30 contiguous nucleotides.

72. The isolated polynucleotide of claim 70, designed to specifically amplify a segment of chromosome 6q23 comprising at least one polymorphic site of the polymorphism indicative of schizophrenia or predisposition to schizophrenia.

73. The isolated polynucleotide of claim 72, wherein the amplified segments of chromosome 6q23 starts at least 15 and not more than 100 nucleotides from a polymorphic site of the polymorphism indicative for schizophrenia or predisposition to schizophrenia.

74. The isolated polynucleotide of claim 69, wherein the nucleotide identity at rs6931735, rs6912933 rs9321501 rs7750586 rs9647635 or rs1475069 is Adenine; the nucleotide identity at rs2746429 is Thymine; the nucleotide identity at rs2614258, rs9494332 or rs797553 is Guanine; and the nucleotide identity at rs11154801, rs7739635, rs642162, rs1414839, or rs1239365 is Cytosine.
